(19) United States
(12) Patent Application Publication
(10) Pub. No.: US 2013/0074556 A1
(43) Pub. Date: Mar. 28, 2013

(54) TETRA VERMI BED AND A PROCESS FOR COMPOSTING AGRICULTURAL WASTE

(71) Applicants: Kishorilal Ramnath Dhoot, Aurangabad (IN); Kamlesh Kishorilal Dhoot, Aurangabad (IN)

(72) Inventors: Kishorilal Ramnath Dhoot, Aurangabad (IN); Kamlesh Kishorilal Dhoot, Aurangabad (IN)

(21) Appl. No.: 13/261,053

(22) PCT Filed: Feb. 6, 2009
(86) PCT No.: PCT/IN2009/000312

§ 371 Date: Sep. 12, 2012

(51) Int. Cl. C05F 17/02; A01K 67/033; C05F 17/00

(52) U.S. Cl. CPC C05F 17/0258; C05F 17/0009; A01K 67/0332
USPC 71/21; 119/6.7

(57) ABSTRACT

A tetra vermi bed and a process for composting agricultural waste The conventional composting apparatus are mostly bulky, posing problems in transportation to sites, storing agricultural waste. This invention addresses the shortcomings by construction of composting bed of multiple layers of LDPE and HDPE substrate. The tetra vermin bed propagates production of worms and yields manure, which is easy to handle and to transport in bags. The tetra vermin bed is easy to install and to dismantle for operation at multiple sites, storing agricultural waste.

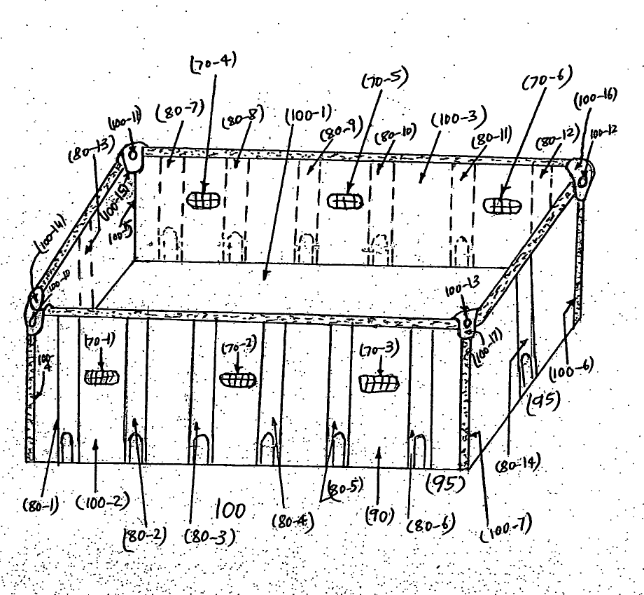

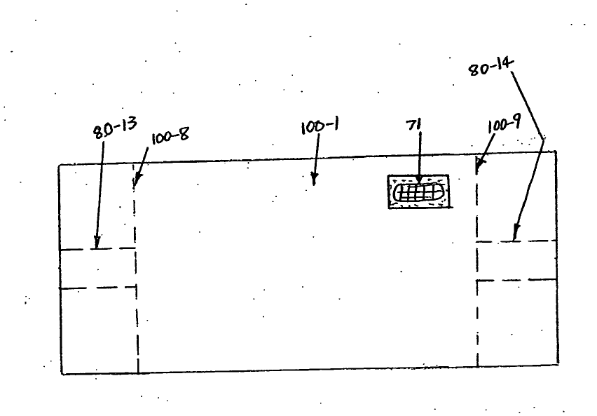

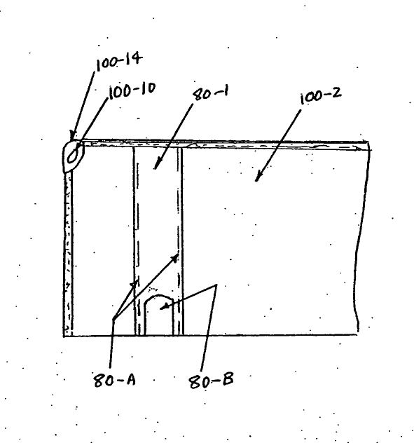

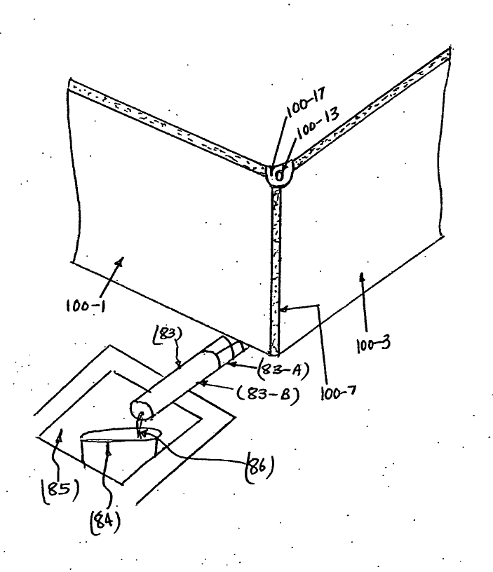

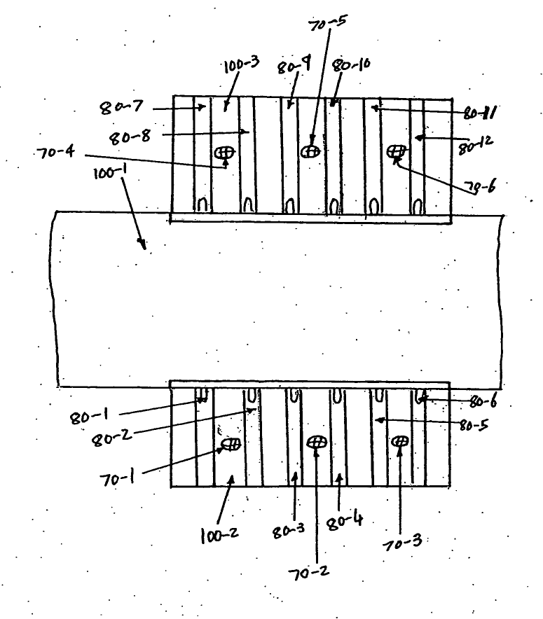

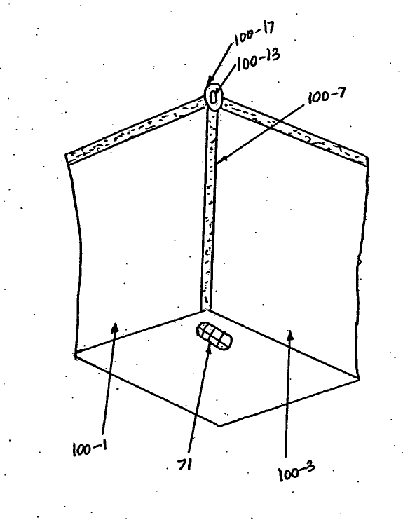

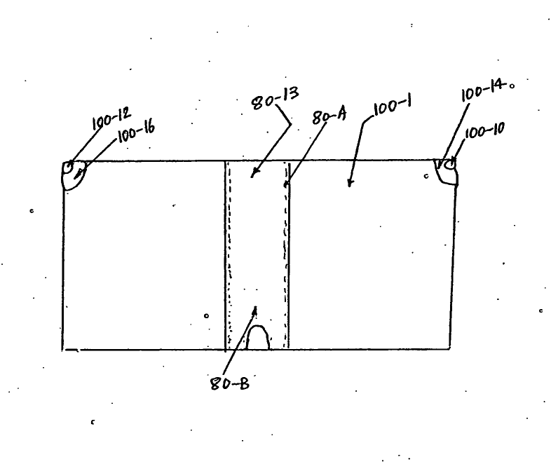

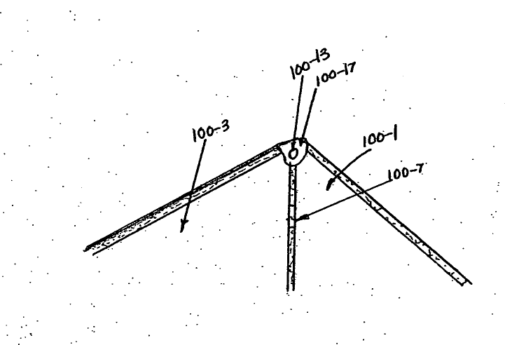

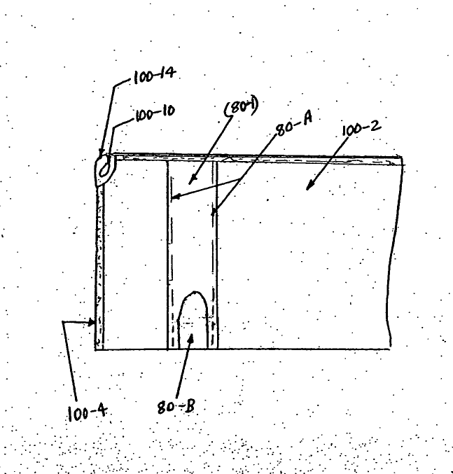

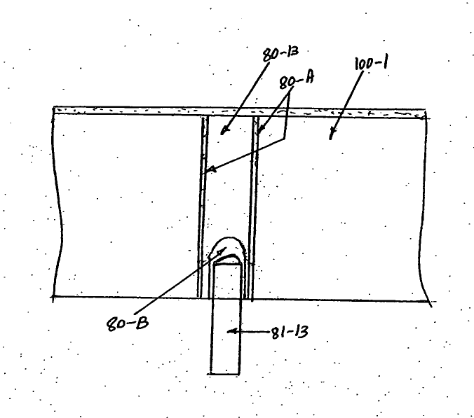

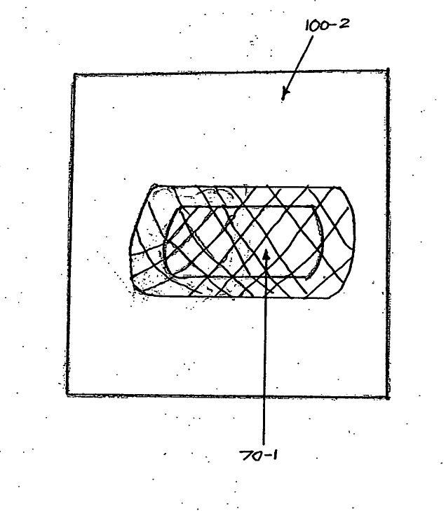

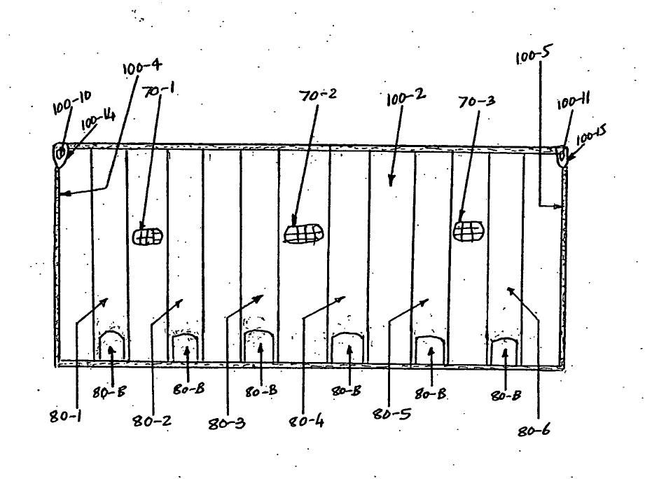

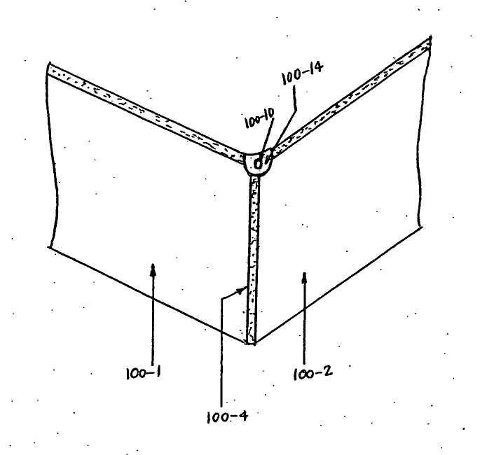

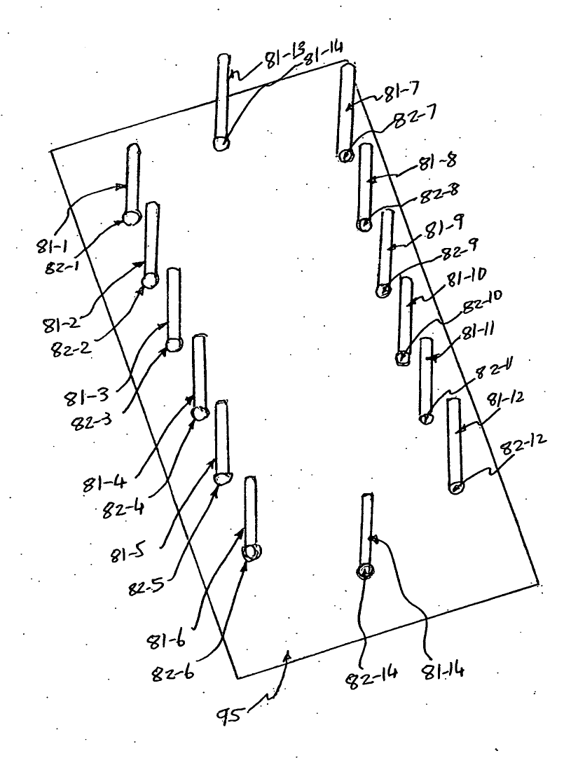

### FIELD OF THE INVENTION

This invention relates to the field of composting waste into manure and more particularly to the field of composting agricultural waste into nutrient-rich manure in an earthworm-friendly, portable, foldable tetra vermi bed, supported on fourteen grounded poles through fourteen totally heat sealed pockets.

### BACKGROUND OF THE INVENTION

Following works are cited to pinpoint inherent drawbacks encountered in composting the agricultural waste.

U.S. Pat. No. 702,952—This work calls for mechanical actions and is operationally complex for semi-literate/illiterate farmers. The present invention requires no energy input.

U.S. Pat. No. 6,488,733—This work entails composting in pits and is restricted to permanently dug pits. The present invention allows portability to site—where agricultural waste is collected—and ease in installation. The present invention is even suitable for sites such as hilly areas which are not easily accessible or pose barriers.

US patent—U.S. Pat. Nos. 11,331,973 and 10,403,816—These works are based on use of animal urine for composting. The composting method and the apparatus are both different from the present invention, which places no reliance on animal urine for composting and yields solid manure for easy end-application.

U.S. Pat. No. 10,872,174—This work relates to composting sewage in an apparatus, using a plurality of tanks and the required piping. The present invention allows portability to site of agricultural waste and requires no plurality of tanks and no complex handling.

U.S. Pat. No. 6,576,462—This work relates to vermi culture composting device for organic/kitchen waste, relying upon sunlight partially to control temperature for composting. Container for composting is painted oppositely in dark and light colours for absorbing or reflecting sunlight. The present invention is entirely different from the cited works in terms of no painting of alternate sides with black shade being required.

FR 2913014—This work employs a plurality of tanks with perforated bottoms and covered with air holes. It employs dendrodrilus rubidus worm for composting. The present invention employs a portable multilayered fabric tank for composting.

U.S. Pat. Des. 332,163 This work employs a vermi composting container, which is fitted with a plurality of perforated pipes near bottom end of the container for introducing water. The present work yields manure, which is easy to handle in end-application and relies upon a composting bed made of a composite fabric.

U.S. Pat. No. 7,018,831—This prior work relates to a composting apparatus which comprises a plurality of stackable composting drawers and a plurality of receivers, each receiver arranged under one unit of said plurality of drawers. The present invention is different from the cited work.

U.S. No. D332,163—This prior work relates to a composting container, which is rigid and employs laterally a plurality of horizontal, perforated, hollow rods. Said rods ends are covered with perforated screen to vent out gases/liquid.

The present invention is different in terms of foldable bed, which—when inserted in fourteen vertically heat-sealed pockets—support the composite fabric bed above ground level in a rectangular shape.

U.S. Pat. No. 6,576,462—This prior work relates to a composting device, which is made of four rectangular plywood rigid panels. The panels exteriorly are painted in varying colors. The present invention employs a portable, foldable bed, which is supported above the ground by means of inserting onto top end of fourteen grounded wooden poles fourteen fully heat-sealed pockets of the foldable bed. The present invention is very much different from the cited work.

### OBJECT OF THE INVENTION

One object of the present invention is to allow portability of the composting apparatus to the sites, having agricultural waste and the ease in installation of the composting apparatus. It is implied that the apparatus is light in weight, easy to carry to the waste cites and quick in installation.

Another object of the present invention is to yield high-quality manure that is easy to handle, to pack in conventional bags and to economically transport.

Another object of the present invention is to propagate reproduction of the culture and to protect the culture against pilferage, predators such as ants, snakes and rodents.

Another object of the present invention is to eliminate, in composting, requirement for mechanical energy and complex, plurality of tanks, vessels and piping system.

### BRIEF DESCRIPTION OF THE DRAWINGS

Drawing #1 illustrates a perspective view of tetra vermi bed (100).

Drawing #2 illustrates main panel (100-1).

Drawing #3 illustrates the corner eyelet (100-10 . . . 100-13)) and flap fold (100-14 . . . 100-17).

Drawing #4 illustrates the installation of vermi wash drain pipe (83) and the dug-out pit (85).

Drawing #5 illustrates joining by heat-sealing the side panels (100-2, 100-3) with the main panel (100-1) and joining two longitudinally extending portions of the main panel (100-1) with the side panels (100-2, 100-3).

Drawing #6 illustrates sealing in vertical configuration of the longitudinally extending portions of the main panel (100-1) with the two lateral sides each of the side panels (100-2, 100-3) and sealing HDPE net over the drain (71).

Drawing #7 illustrates inverted U-shaped pocket (80-13, 80-14) of main panel (100-1) and half-rounded opening at bottom to facilitate insertion of wooden pole (81)

Drawing #8 illustrates interior view of vertical corner joint (100-4 . . . 100-7).

Drawing #9 illustrates pocket U-cutting of main panel (100-1) and main panel (100-1) top edge rope folding.

Drawing #10 illustrates inserting tetra vermi bed (100) onto grouted wooden pole (81-1 . . . 81-14).

Drawing #11 illustrates HDPE net sealing over window cutting (70-1 . . . 70-6).

Drawing #12 illustrates inverted-U shaped pockets (80-1 . . . 80-12), windows (70-1 . . . 70-6) heat sealed on side panel (100-2, 100-3).

Drawing #13 illustrates side panel (100-2, 100-3) vertical sealing (100-4 . . . 100-7)

Drawing #14 illustrates fourteen wooden poles (81-1 . . . 81-14) grouted in dug-out holes (82-1 . . . 82-14) in ground (95) respectively.

### DETAILED DESCRIPTION OF THE INVENTION

Tetra vermi bed (100) is constructed of a seven-layered, chemically-treated, U.V.-stabilized, composite HDPE woven fabric (90), which is prepared by alternately laminating a layer of LDPE (92) in between two layers of HDPE (91). The composite fabric (90) has three layers of HDPE, which are made from HDPE granules, added with UV-stabilizing compound, and which alternate with four LDPE layers (signifying the word ‘Tetra’ of the product's name). The combined seven layers are laminated by extrusion coating into a single composite fabric (90). AN INVENTIVE FEATURE OF THE PRESENT INVENTION IS THE USE OF THE UV-STABILIZED, COMPOSITE FABRIC (90) IN CONSTRUCTION OF TETRA VERMI BED (100). UV-stabilizing compound is added 3-5% by weight to the granules to prevent cracking of the composite fabric (90), caused due to exposure to sunlight.

Tetra vermi bed (100) is constructed of a main panel (100-1) of said composite fabric (90), admeasuring 16 feet 9 inches by 4 feet 2 inches and of a twin side panel (100-2, 100-3) of said composite fabric (90), admeasuring 12 feet 9 inches by 2 feet. Each side panel (100-2, 100-3) is processed, as explained below, before it is joined with the main panel (100-1):

Tetra vermi bed (100) is firmly erected over laid ground (95)—provided with a gradient of 6 inches over 12 feet—by means of fourteen pockets (80-1 . . . 80-14), of which six pockets (80-1 . . . 80-6) exteriorly and perpendicularly to longitudinal axis are heat-sealed on the side panel (100-2) of the tetra vermi bed (100), as illustrated in the accompanying drawing #1, and the other six pockets (80-7 . . . 80-14) exteriorly and perpendicularly to the longitudinal axis are heat-sealed on the other side panel (100-3) of the tetra vermi bed (100). The thirteenth pocket (80-13) exteriorly and parallel to the longitudinal axis is heat-sealed, as illustrated in the accompanying drawing #1, on one lateral side of the main panel (100-1) of the tetra vermi bed (100) and the fourteenth pocket (80-14) exteriorly and parallel to the longitudinal axis is heat-sealed on the other lateral side of the main panel (100-1) of the tetra vermi bed (100). The fourteen pockets (80-1 to 80-14) are of inverted U-shape, open at bottom end and closed at the top end such that straight, 1^{1/2} inch diameter wooden pole (81) is easily slid through 6 inch clearance provided in each of said fourteen pockets (80-1 to 40-14) for the firm installation of the tetra vermi bed (100) over the well-laid ground (95). THE SECOND INVENTIVE FEATURE OF THE PRESENT INVENTION IS THE FOURTEEN, INVERTED U-SHAPED POCKETS (80-1 TO 80-14), AS EXPLAINED ABOVE.

Each U-shaped pocket (80-1 to 80-14) is—during process of attachment—heat-sealed simultaneously on two sides thereof (80A) onto the panels (100-1, 100-2, 100-3) of the tetra vermi bed (100), leaving a six inch clearance (80B) along the entire length of the U-shaped pocket being attached (80-1 to 80-14), as illustrated in the accompanying drawings #7, 9 and 12. THE THIRD INVENTIVE FEATURE IS CHARACTERIZED BY THE TWIN-SIDED HEAT-SEALING (80A) OF FOURTEEN POCKETS (80-1 TO 80-14) AND ALLOWING SIX INCH CLEARANCE (80B) ALONG THE ENTIRE LENGTH OF THE FOURTEEN POCKETS (80-1 TO 80-14).

Tetra vermi bed (100) breathes by ventilating out hot air generated within the mass being composted and facilitating circulation of fresh air into the composting mass through six (70-1 . . . 70-6) windows. Three windows (70-1 . . . 70-3) are provided on the side panel (100-2) of the tetra vermi bed (100) and the other three windows (70-4 . . . 70-6) are provided on the other side panel (100-3) of tetra vermi bed (100). The windows (70-1 . . . 70-6) are marked on the respective side panels (100-2, 100-3) of the tetra vermi bed (100) and the marked areas with round corners are cut out from the respective side panels. An HDPE net is sealed onto the cut portion, as illustrated in the accompanying drawing #11.

A drain (71) is provided in the main panel (100-1) of the tetra vermi bed (100), as illustrated in the accompanying drawing #4, by marking, cutting out the marked portion and sealing with an HDPE net (71). THE FOURTH INVENTIVE FEATURE OF THE PRESENT INVENTION IS CHARACTERIZED BY THE PROVISION OF SIX WINDOWS (70-1 . . . 70-6)—IN THE FORM OF HDPE NET—ON THE SIDE PANELS (100-2, 100-3) AND A DRAIN OUTLET (71)—IN THE FORM OF HDPE NET—ON THE MAIN PANEL (100-1), AS EXPLAINED ABOVE.

The side panels (100-2, 100-3) and the main panel (100-1) have their respective longitudinal and the lateral ends—farthest to the U-shaped mouths of the pockets (80-1 to 80-14)—roped end-to-end. ROPING THE MAIN PANEL (100-1) AND THE SIDE PANELS (100-2, 100-3) CHARACTERIZE THE FIFTH INVENTIVE FEATURE OF THE TETRA VERMI BED (100), AS EXPLAINED ABOVE.

The inverted U-shaped pockets (80-1 to 80-12), after heat sealing with the side panels (100-2, 100-3), have their other end cut into U-shape, folded over the roped edge of the respective panels (100-2, 100-3) for anchoring and heat-sealed to the interior side of the panels (100-2, 100-3). U-pockets (80-13, 80-14) are similarly anchored over the two lateral sides of the main panel (100-1). ANCHORING THE INVERTED, U-SHAPED POCKETS (80-1 . . . 80-14) OVER THE PANELS (100-1 . . . 10-3), AS EXPLAINED ABOVE, CHARACTERIZE THE SIXTH INVENTIVE FEATURE OF THE TETRA VERMI BED (100).

The side panels (100-2, 100-3) at the un-roped longitudinal ends are heat-sealed with the main panel (100-1), thereby anchoring the twelve U-pockets (80-1 . . . 80-12) at their respective lower ends, as illustrated in the accompanying drawing #5. The two lateral sides of the side panels (100-2, 100-3) are sealed in vertical configuration with the respective extending sides of the main panel (100-1) to form four joints (100-4 to 100-7), that form the four vertical sides of the tetra vermi bed (100). The vertical joints (100-4 . . . 100-7) are each folded sideways and then heat-sealed over the folds to impart extra strength against tear, as illustrated in the accompanying drawing #6. THE SEVENTH INVENTIVE FEATURE OF THE TETRA VERMI BED IS CHARACTERIZED BY THE JOINING, BY HEAT-SEALING, THE TWO SIDE PANELS (100-2, 100-3) WITH THE MAIN PANEL (100-1) AND STRENGTHENING THE JOINTS—THAT FORM THE VERTICAL SIDES OF THE TETRA VERMI BED (100)—BY FOLDING SIDEWAYS THE VERTICAL JOINTS AT (100-4 TO 100-7) AND HEAT-SEALING OVER THE FOLDS THE FOUR VERTICAL JOINTS (100-4 TO 100-7).

The tetra vermi bed (100) is fully constructed in a foldable form and is ready for installation.

The novelty of the present invention lies in the installation of the tetra vermi bed (100) by grouting fourteen straight, 40 inch-long, circular wooden poles (81-1 to 81-14) to 18 inches depth from the ground level (95)—as illustrated in the accompanying drawing #14—sliding downward the fourteen pockets (80-1 . . . 80-14) over the grouted fourteen wooden poles (81-1 . . . 81-14) to the points closest to the ground surface such that the poles (81-1 . . . 81-14) firmly hold the four vertical sides of the tetra vermi bed (100), with the central portion of the main panel (100-1) forming the bottom of the tetra vermi bed (100) between the two fold lines (100-8, 100-9), as illustrated in the accompanying drawing #2.

Four eyelets (100-10 to 100-13) are provided at top four corner points of the tetra vermi bed (100) to facilitate roping and the eyelets are covered with flaps (100-14 to 100-17), as illustrated in the accompanying drawing #3, to protect the roping from damage by the edges of the eyelets (100-10 to 100-13).

A drain (71) is fabricated at the marked portion of the main panel (100-1) by cutting out the marked portion with round corners and heat-sealing an HDPE net over the cut portion. A HDPE drain pipe (83), semi-circularly cut-open along a part length, is fitted under the drain (71) such that the tetra vermi bed (100) fits neatly over the cut-open length (83-A) of the pipe (72) and the remainder uncut portion (83-B) is buried in the ground and placed above the container (84), as illustrated in the accompanying drawing #4; which is placed in the dug-out pit (85) to collect vermi wash liquid (86). The drain (71) is positioned at the lowest point of the gradient of the laid ground (95). THE EIGHTH INVENTIVE FEATURE OF THE PRESENT INVENTION IS CHARACTERIZED IN THE CONSTRUCTION OF THE DRAIN (71) AND BY THE POSITIONING OF THE DRAIN PIPE (83), SEMI-CIRCULARLY CUT OPEN ALONG A PART LENGTH (83-A) FOR PLACEMENT UNDER THE DRAIN (71) OF THE TETRA VERMI BED (100).

One embodiment of the present invention is constructed along the following technical parameters of specific values, as summarized below, for the tetra vermi bed (100) of size 12 feet×4 feet×2 feet to perform optimally. They are as follows:

Main Panel:

| | | | |
|---|---|---|---|
| 1. | | inverted U-pockets (80-13, | 2 number, size-24 inches × 8 |
| | | inches 80-14) | |
| 2. | | drain (71) | 3.5 inch × 1.5 inch |
| 3. | | top edge rope folding | 2 inch |
| 4. | (a) | main panel (100-1) to side } | 2 inch |
| | | panel (100-2, 100-3) sealing } | |
| | | width } | |
| | (b) | four end vertical joint } | 2 inch |
| | | sealing, formed between } | |
| | | lateral end of side panel } | |
| | | (1002; 100-3) and } | |
| | | extended portion of main } | |
| | | panel (100-1) } | |
| | (c) | fold sealing width } | 2 inch |

Side panel:

| | | |
|---|---|---|
| 5. | inverted U-pockets (80-1... | 24 inch × 8 inch |
| | 80-12) | |
| 6. | window (70-1...70-6) | 8 inch × 4.5 inch |
| 7. | pocket to window distance | 4.5 inch |
| 8. | vertical corner-to-pocket | 5 inch |
| | distance | |
| 9. | pocket U-cut | 3.5 inch × 4.5 inch |
| 10. | top edge rope fold | 2 inch |
| 11. | colour | exterior-green, interior-white |
| 12. | tensile strength of the } | warp - 190 kgf |
| | composite fabric (90) } | weft - 125 kgf |
| 13. | tensile strength at bottom | 100 kgf |
| | Seal | |
| 14. | tensile strength at pocket seal | 100 kgf |
| 15. | tear strength of fabric | warp - 200 kgf |
| | | weft - 140 kgf |
| 16. | bursting strength of fabric | 35 kg/sq. cm. |
| 17. | water penetration | should pass |

(specifications 11 to 17 apply to main panel (100-1), side
panels (100-2, 100-3) and pockets (80-1...80-14) )

| | | |
|---|---|---|
| 18. | accelerated aging test } | should pass |
| 270 | at 70 deg. C., 7 days } | should pass |
| 19. | chemical resistance test | should pass |
| 20. | environment stress and } | |
| | resistance to cracking } | |
| | -at 50 degree C., 24 hours | should pass |
| | -at 90 degree C., 48 hours | should pass |

We claim:

1. A stitch less, heat-sealed, HDPE substrate-tetra LDPE-layered, vermi composting bed (100), constructed of a composite fabric (90), said composite fabric (90) made of UV-stabilized, four, hot extrusion coated, LDPE layers, said four layers laminated and alternating with UV-stabilized, three, HDPE substrate (woven fabric) layers, said tetra vermi bed (100) comprising:
(i) a main panel (100-1), of said composite fabric (90), admeasuring 16 feet, 9 inches×2 feet;
(ii) two side panels (100-2, 100-3), each said side panel of said composite fabric (90), admeasuring 12 feet 9 inches×2 feet such that
(iii) (a) the ultimate size of said tetra vermi bed (100), made of said seven-layered composite fabric (90) admeasures 12 feet long 4 feet wide×2 feet high;
(b) said vermi bed (100) has an internal volumetric capacity to contain 96 cubic feet volume of waste; and
(c) the ultimate shade of said tetra vermi bed (100) exteriorly is green and interiorly is snow white;
(iv) fourteen, inverted U-shaped pockets (80-1 . . . 80-14) made of said seven-layered composite fabric (90), each said pocket open with inverted U-shaped mouth at bottom end thereof and closed at top end thereof by anchoring by roped end of said side panels (100-2, 100-3) or of said main panel (100-1);
(v) fourteen round, straight, 39 inch long wooden poles (81-1 . . . 81-14), each said pole grouted at a pre-marked holes (82-1 . . . 82-14) to 18 inches depth below the laid ground surface (95) and projecting above the ground surface (95) to a height of 21 inches;
(vi) six windows (70-1 . . . 70-6), made by cutting out respective pre-marked portion from said side panels (100-2, 100-3) and heat sealing HDPE net, over said cutout portions with round corners, each said window admeasuring 8 inches×4.5 inches;
(vii) a drain (71), cut open in marked portion of said main panel (100-1) and covered with a HDPE net over the drain (71) opening, said drain (71) resting atop a semi-circularly, cut-open pipe (83) along part length (83-A) and buried for the remainder length (83-B) in soil under said drain (71) of said tetra vermi bed (100), said drain (71) admeasuring 3.5 inches×1.5 inches.

2. A stitch less, heat-sealed, HDPE substrate-tetra LDPE-layered, vermi composting bed (100) as claimed in claim 1, wherein said six pockets (80-1 . . . 80-6)
(a) admeasure 24 inches×8 inches; and
(b) are heat-sealed on said green exterior side of said side panel (100-2) in a position, perpendicular to longitudinal axis of said side panel (100-2) of said tetra vermi bed (100).

3. A stitch less, heat-sealed, HDPE substrate-LDPE-layered, vermi composting bed (100) as claimed in claim 1, wherein the other said six pockets 80-7 . . . 80-12)
(a) admeasure 24 inches×8 inches; and
(b) are heat-sealed on said green exterior side of said side panel (100-3) in a position, perpendicular to longitudinal axis of said side panel (100-3) of said tetra vermi bed (100).

4. A stitch less, heat-sealed, HDPE substrate-tetra LDPE-layered, vermi composting bed (100) as claimed in claim 1, wherein said thirteenth pocket (80-13)
(a) admeasures 24 inches×8 inches; and
(b) is heat-sealed on said green exterior surface and near one lateral side of said main panel (100-1), in position parallel to the longitudinal axis of said main panel (100-1).

5. A stitch less, heat-sealed, HDPE substrate-tetra LDPE-layered, vermi composting bed (100) as claimed in claim 1, wherein said fourteenth pocket (80-14)
(a) admeasures 24 inches×8 inches; and
(b) is heat-sealed on said green exterior surface and near said other lateral side of said main panel (100-1), in position parallel to the longitudinal axis of said main panel (100-1).

6. A stitch less, heat-sealed, substrate-HDPE-tetra LDPE-layered, vermi composting bed (100) as claimed in claim 1, wherein said windows (70-1 . . . 70-3) are centrally cut open from said side panel (100-2) in 8 inch×4.5 inch openings with round corners which are heat-sealed with HDPE nets.

7. A stitch less, heat-sealed, HDPE substrate-tetra LDPE-layered, vermi composting bed (100) as claimed in claim 1, wherein said windows (70-4 . . . 70-6) are centrally cut open from said side panel (100-3) in 8 inch×4.5 inch openings with round corners which are heat-sealed with HDPE nets.

8. A stitch less, heat-sealed, HDPE substrate-tetra LDPE-layered, vermi composting bed (100) as claimed in claims 1 and 2 to 5, wherein said pockets (80-1 . . . 80-14) are heat-sealed on two sides (80-A) of said pockets (80-1 . . . 80-14) over a 6 inch clearance (80-B) along the entire length of said pockets (80-1 . . . 80-14).

9. A stitch less, heat-sealed, HDPE substrate-tetra LDPE-layered, vermi composting bed (100) as claimed in claim 1, wherein
(a) said side panels (100-2, 100-3) are rope-folded by 2 inches at one longitudinal end, that is farthest from said inverted, U-shaped mouth of said pockets (80-1 . . . 80-12); and
(b) said main panel (100-1) is rope-folded by 2 inches at both lateral ends, which are farthest to said inverted, U-shaped mouth of said pockets (80-13, 80-14).

10. A stitch less, heat-sealed, HDPE substrate-tetra LDPE-layered, vermi composting bed (100) as claimed in claim 1, wherein
(i) said each side panel (100-2, 100-3) at un-roped longitudinal side is heat sealed with one longitudinal side of said main panel (100-1)
(ii) two lateral sides of each side panel (100-2, 100-3) are each heat-sealed with extended longitudinal sides of said main panel (100-1) to form vertical corner joints (100-4 . . . 100-7) that are folded sideways by 2 inches and sealed over folds for additional strength.

11. A stitch less, heat-sealed, HDPE substrate-tetra LDPE-layered, vermi composting bed (100) as claimed in claim 1, wherein
(i) said window (70-1, 70-2, 70-3, 70-4, 70-5, 70-6) each is 4.5 inch away from said adjoining, inverted U-shaped pocket [(80-1, 80-2), (80-3,80-4), (80-5, 80-6), (80-7,80-8), (80-9, 80-10), (80-11,80-12)] respectively and
(ii) said inverted U-shaped pocket (80-1, 80-6, 80-7, 80-12) each is 5 inch away from said vertical corner joint (100-4, 100-5, 100-6, 100-7) respectively.

12. A stitch less, heat-sealed, HDPE substrate-tetra LDPE-layered, vermi composting bed as claimed in claims 1, 2 and 3, wherein
(a) each said inverted U-shaped pocket (80-1 . . . 80-12) at U-shaped one end is heat-sealed—along with said side panel (100-2, 100-3)—to said main panel (100-1) and at said closed other end is anchored over the roped top side of said side panels (100-2, 100-3) by cutting out a U-shaped portion off the closed other end and heat sealing it with said side panel (100-2, 100-3);
(b) each said inverted U-shaped pocket (80-13, 80-14) at closed other end is anchored over the roped top side of said main panel (100-1) by cutting out a U-shaped portion off the closed other end and heat sealing it with said main panel (100-1).

13. A stitch less, heat-sealed, HDPE substrate-tetra LDPE-layered, vermi composting bed (100), as claimed in claim 1, is installed on well laid ground (95) having a gradient of 6 inches over 12 feet by
(i) marking correctly fourteen locations for grouting said fourteen wooden poles (81-1 . . . 81-14) on an area selected for installation, said selected area of said well-laid ground (95) admeasuring 15 feet×6 feet;
(ii) digging said marked positions into fourteen holes (82-1 . . . 82-14) each said hole admeasuring 3 inches in diameter×18 inches deep;
(iii) grouting each said wooden pole (81-1 . . . 81-14) in each said dug hole (82-1 . . . 82-14) such that each said pole (81-1 . . . 81-14) rise above said well-laid ground (95) to a height of 21 inches;
(iv) inserting said pockets (80-1 . . . 80-14) onto said respective wooden poles (81-1 . . . 81-14) of said tetra vermi bed (100) simultaneously and sliding down gradually said tetra vermi-bed (100) over said wood wooden poles (81-1 . . . 81-14) till said tetra vermi bed (100) touches said well laid ground (95) all along its periphery.
(v) fitting under said drain (71) said drain pipe (83) such that said tetra vermi bed (100) sits atop said semi-circularly cut-open portion (83-A) of said drain pipe (83) and placing the other end (83-B) of said drain pipe (83) above a container (84), placed in a dug-out pit (85) for collecting vermi wash liquid (86).

14. A process for composting agricultural waste in a tetra vermi bed (100) comprising:
(i) sorting out hard material, including stones and domestic animal dung from partly decomposed agricultural waste and leaching out animal urine from the partly decomposed waste by watering and precipitating out salt to less than 0.5% by weight level;
(ii) layering alternately in the tetra vermi bed said partly decomposed waste and said animal dung each in 6 inch thick layers and repeating said layering process till the tetra vermi bed is almost filled to brim or alternatively filling completely with animal dung;
(iii) sprinkling water in fine spray over the contents of the tetra vermi bed till heat of the contents is positively removed and the contents of the tetra vermi bed are cooled and humidity initially is 70%-80% by weight;
(iv) checking settling down of the contents after sprinkling water and adding a layer of animal dung till the contents reach the 2 feet height of the tetra vermi bed;
(v) measuring periodically temperature of the contents and maintaining it to 25 degree C. to 35 degree C. and maintaining moisture to adequate levels;
(vi) periodically checking pH value and maintaining it to 5 to 9 level;
(vii) adding calcium carbonate to the contents to raise pH to 5 to 9 level or adding peat moss to the contents to lower pH to the required 5-9 level, ideal pH being 7.5 indicating slightly alkaline nature, favourable for reproduction of worms.
(viii) making 3 inch-4 inch deep basins into the contents along all sides of the tetra vermi bed, after it is sufficiently cooled down and the water content comes down to 20-30% by weight so as the waste to become sufficiently moist and porous for free movement of earthworms;
(ix) adding earthworms to said basins, allowing them to mix with the contents and closing said basins;
(x) repeating the process after three days, as described in step (viii) above but to add proper feed for worms;
(xi) frequently watering the contents for maintaining the porousness of the decomposed waste to facilitate free movement of worms;
(xii) allowing earthworms for a period of 2 months to work on the partly-decomposed agricultural waste;
(xiii) collecting vermi wash liquid in the container placed in the pit;
(xiv) periodically inspecting said vermi wash liquid, earthworms and the tetra vermi bed and monitoring temperature;
(xv) gathering the produced manure, after 40 days, from all sides of the tetra vermi bed and stacking it in a pile, leaving thereby earthworms at bottom of the tetra vermi bed;
(xvi) sieving the produced manure and packing it in bags;
(xvii) sampling the packed manure for inspection for quality check.
(xviii) re-using earthworms segregated from the produced manure in following manure production batches.

15. A stitch less, heat-sealed, HDPE substrate-tetra LDPE-layered, vermi composting bed (100), as claimed in claim 1, wherein four eyelets (100-10 to 100-13) are provided at top four corner points of said tetra vermi bed (100) for roping the top edges of said side panels (100-2, 100-3) and of said main panel (100-1), said eyelets (100-10 . . . 100-13) being covered with folding flaps (100-14 to 100-17) to protect the roping from damage by the edges of said eyelets (100-10 to 100-13).

16. A tetra vermi bed and a process for composting agricultural waste into manure, as substantially described herein above and illustrated in the accompanying 1 to 14 drawings
